(19) United States
(12) Patent Application Publication
(10) Pub. No.: US 2013/0316010 A1
(43) Pub. Date: Nov. 28, 2013

(54) POLYMERIC MICROPARTICLES

(71) Applicant: Erin Lavik, Cleveland Heights, OH (US)

(72) Inventor: Erin Lavik, Cleveland Heights, OH (US)

(73) Assignee: Case Western Reserve University, Cleveland (OH)

(21) Appl. No.: 13/879,981

(22) PCT Filed: Oct. 18, 2011
(86) PCT No.: PCT/US11/56732

§ 371 Date: Jul. 29, 2013

Related U.S. Application Data
(60) Provisional application No. 61/394,126, filed on Oct. 18, 2010.

(51) Int. Cl. A61K 9/14; A61K 31/519; A61K 31/573; A61K 31/5377

(52) U.S. Cl. CPC A61K 9/146; A61K 31/5377; A61K 31/519; A61K 31/573
USPC 424/501; 514/236.2; 514/249; 514/180

(57) ABSTRACT

A pharmaceutical composition is provided comprising microparticles encapsulating high weight percent active agent and providing sustained release over a prolonged period of time of active agent levels bioequivalent to direct administration of active agent. Polymeric microparticle compositions containing one or more active agents, and methods of making and using thereof, are described. The microparticles are optimized for the agent to be delivered, so that the hydrophobicity or hydrophilicity of the polymer and charge of the polymer maximizes loading of the agent, and the selection and molecular weight of the polymers maximize release of an effective amount of the active agent for the desired period of time.

This application claims priority of U.S. provisional patent application No. 61/394,126 filed Oct. 18, 2010, the disclosure of which is incorporated in its entirety herein.

### FIELD OF THE INVENTION

A pharmaceutical composition is provided comprising microparticles encapsulating high weight percent active agent and providing sustained release over a prolonged period of time of active agent levels bioequivalent to direct administration of active agent.

### BACKGROUND OF THE INVENTION

Polymeric microparticles have been used for active agent delivery for decades. Numerous methods to increase the amount of drag which can be delivered, and to manipulate rate of release, and release profile, have been described. Methods have included altering microparticle size, shape, polymer composition, inclusion of additives such as surfactants and pore forming agents, and inclusion of ligands and bioadhesive agents.

Glaucoma is an ophthalmic disease characterized by the gradual degeneration of retinal ganglion cells (RGCs). RGCs synapse with bipolar cells and transmit visual inputs to the brain along the optic nerve. Degeneration of these cells leads to gradual vision loss and ultimately blindness if untreated. Glaucoma is the second leading cause of blindness (Biomdahl et al, Acta. Opth. Scan., 75, 310-319 (1997)). Glaucoma will affect approximately 60.5 million people in 2010, increasing to 796 million people in 2020 (Quigley et al, Brit, J. Opth, 90, 262-267 (2006)). This includes peoples suffering from both open angle (OAG) and angle closure glaucoma (ACG).

Although a normal tension variant does exist, the development of glaucoma is most often associated with elevated intraocular pressure (TOP) (Migdal et al., Opthmal, 101, 1651-1656 (1994)). This elevated pressure is caused by an excess accumulation of aqueous humor in the eye due to blockage of the trabecular network (Alward et at, Amer. J. Opthmal., 126, 498-505 (1998)). With a majority of glaucoma cases associated with elevated IOP, reduction of this pressure has been found to greatly mitigate degeneration in approximately 90% of the cases, including cases in which IOP is in the normal range but optic neuropathy occurs (Id).

Eye drops containing one more active agent that lower IOP are typically prescribed to treat glaucoma, are currently the primary means of delivery for this active agent. However, eye drop typically deliver very small amounts of active agent, requiring large numbers of doses per day for IOP management. Compliance with this treatment regime is poor with more than half of patients unable to maintain consistently lowered IOP through drops (Rotchford and Murphy, Brit. J Opthmal, 12, 234-236 (1998)).

Drops also lead to extensive systemic absorption of the administered active agent (−80%, Marquis and Whitson, Active agents & Aging, 22, 1-21 (2005)). This systemic absorption can result in adverse side effects. Together, these complications make topical application of IOP-lowering active agents problematic, especially in the aging population that exhibits the lowest compliance and highest degree of complications (Marquis and Whitson, Active agents & Aging, 22, 1-21 (2005)). There exists a need for sustained release formulations, which overcomes the limitations of currently available eye drops.

A variety of approaches for the sustained delivery of active agents to the have been investigated

U.S. Pat. No. 6,726,918 to Wong describes methods for treating inflammation-mediated conditions of the eye, the methods including implanting into the vitreous of the eye a bioerodible implant containing a steroidal anti-inflammatory and a bioerodible polymer, wherein the implant delivers an agent to the vitreous in amount sufficient to reach a concentration equivalent to at least about 0.65 μg/m1 dexamethasone within about 48 hours and maintains a concentration equivalent to at least about 0.03 μg/ml dexamethasone for at least about three weeks. Wong does not disclose administering the implants by subconjunctive injection. U.S. Patent Application Publication No. 2006/0173060 to Chang et al. describes biocompatible microparticles containing an alpa-2-adrenergic receptor agonist and a biodegradable polymer. The microparticles can allegedly be used to treat glaucoma. Chang alleges that the microparticles release the active agent for a period of time of at least about one week, such as between two and six months. Chang discloses that the microparticles can be administered subconjunctivally.

U.S. Patent Application Publication No. 2004/0234611 to Ahlheim et al. describes an ophthalmic depot formulation containing an active agent embedded in a pharmacologically acceptable biocompatible polymer or a lipid encapsulating agent for periocular or subconjunctival administration. The formulation can be in the form of microparticles. Ahlheim discloses that the depot formulations are adapted to release all or substantially all of the active material over an extended period of time (e.g., several weeks up to 6 months). Suitable active agents are listed in paragraphs 0033 to 0051; however, the preferred active agent is a stauiosporine, a phthalazine, or a pharmaceutically salt thereof. Suitable polymers are listed in paragraphs 0014 to 0026. Ahlheim contains no examples showing in vitro or in vivo release of any active agents.

None of the references discussed above disclose optimizing the charge, hydrophilicity or hydrophobicity, and/or the molecular weight of the polymers used to prepare the microparticles in order to maximize active agent loading and release of an effective amount of the active agent for a desired period of time.

Thus, there exists a need in the art to provide improved sustained release polymeric microparticle compositions which have been further optimized to maximize active agent loading and release an effective amount of a drag (or active agents) for a desired period of time.

### SUMMARY OF THE INVENTION

Polymeric microparticle compositions containing one or more active agents, and methods of making and using thereof, are described. The microparticles are optimized for the agent to be delivered, so that the hydrophobicity or hydrophilicity of the polymer and charge of the polymer maximizes loading of the agent, and the selection and molecular weight of the polymers maximize release of an effective amount of the active agent for the desired period of time. For example, poorly water soluble active agents tend to interact more strongly with hydrophobic monomers or polymers.

In various embodiments, the microparticle compositions are useful for managing elevated intraocular pressure (TOP), to promote regeneration of the optic nerve and/or for treating one or more anti-inflammatory diseases, such as uveitis.

The microparticle compositions release an effective amount of the active agent for a period greater than 14 days in vivo, greater than 30 days, greater than 60 days in vivo, greater than 75 days in vivo, greater than 90 days in vivo, greater 100 days in vivo, or greater than 120 days in vivo. The desired amount and duration of release is dependent upon several factors including the disease or disorder to be treated, the active agent to be delivered, and the frequency of administration.

In various embodiments, the microparticles are formed from polylactide-co-glycolide (“PLGA”) and polylactic acid (“PLA”). Higher molecular weight polymers, having different ratios of lactic acid (LA), which has a longer degradation time, up to one to two years, to glycolic acid (GA), which has a short degradation time, as short as a few days to a week, are used to provide release over a longer period of time. The combination of active agent loading and release rate, as well as the minimization of initial burst release, result in prolonged release of a higher amount of the active agent. The sustained release of active agent, in combination with the ability to administer the active agent in a minimally invasive manner, leads to improved patient compliance.

The percent loading of the active agent in the microparticles is from about 1% to about 25% by weight, from about 1% to about 20% by weight, or from about 1% to about 10% by weight. The percent loading is dependent on the active agent to be encapsulated, the polymer or polymers used to form the microparticles, and/or the procedure used to prepare the microparticles.

The microparticle compositions are administered to the eye using a variety of techniques in the art. In one embodiment, the compositions are administered to the eye by injection. In another embodiment, the microparticle composition is administered subconjunctival. Subconjunctival administration is minimally invasive, and minimizes systemic absorption of the active agents.

Accordingly, a composition is provided comprising biodegradable microparticles comprising poly(D,L-lactic acid) (PLA) at a weight percent of less than 50% and a second polymer, the microparticles further comprising an ocular therapeutic agent, the microparticles having an average diameter of between about 5 microns and about 50 microns, the microparticles having a structure such that the therapeutic agent is released in a detectable amount from the microparticles for more than 90 days in vivo.

In various aspects, the microparticles in the composition have a structure such that the therapeutic agent is released in a detectable amount from the microparticles for more than 100 days in vivo, more than 120 days in vivo or more than 150 days in vivo. In various aspects, the microparticles comprise PLA at a weight percent of at least 5%, at least 10%, at least 15%, at least 20%, at least 25%, at least 30%, at least 35%, at least 40%, or at least 45%. In various aspects, the PLA has a molecular weight of about 10 kDa to about 50 kDa, about 15 kDa to about 45 kDa, about 20 kDa to about 40 kDa, about 20 kDa to about 35 kDa, or about 20 kDa to about 30 kDa. In one aspect, the PLA has a molecular weight of about 25 kDa.

In various aspects, the composition comprises microparticles that further comprise poly(lactic-co-glycolic acid) (PLGA). In various aspects, the PLGA has a lactic acid to glycolic acid ratio of about 50:50 to about 75:25. In various aspects, the PLGA is about 10 kDa, about 15 kDa, about 20 kDa, about 25 kDa, about 30 kDa, about 35 kDa, about 40 kDa, about 45 kDa, or about 50 kDa.

In various aspects, the therapeutic agent is hydrophobic.

In various aspects, of the composition, the therapeutic agent is an agent that lowers intraocular pressure, an antibiotic, an inhibitor of a growth factor receptor, a chemotherapeutic agent, an anti-inflammatory agent or a steroid. In specific aspects, the therapeutic composition is timolol, methotrexate, or triamcinolone.

In various aspects, the composition is injectable through a 30 gauge needle under clinically acceptable conditions of time and force.

In various aspects, the composition comprises microspheres which comprise about 30% PLA and about 70% PLGA, the PLA has a molecular weight of about 25 kDa and the PLGA has a molecular weight of about 10 kDa, the PLGA has a lactic acid to glycolic acid ratio of about 50:50, and the therapeutic agent is hydrophobic.

A method of treating an ocular condition is also provided comprising the step of administering the composition of the disclosure to a patient in an amount effective to treat the condition. In various aspects, the ocular condition is selected from the group consisting of glaucoma, diabetic retinopathy, age-related macular degeneration, and uveitis. In various aspects, the method of the disclosure comprises administration which is intravitreal or subconjunctival.

### DETAIL DESCRIPTION OF THE INVENTION

### I. DEFINITIONS

“Microparticle,” as used herein, unless otherwise specified, generally refers to a particle of a relatively small size, but not necessarily in the micron size range, and are in various aspects, from less than 50 nm up to 100 microns or greater. “Microparticles,” in various aspects, refers to particles having a diameter from about 5 to about 25 microns, from about 10 to about 25 microns, or from about 10 to about 20 microns. As used herein, the microparticle encompasses microparticles, microcapsules and microparticles, unless specified otherwise. A microparticle is, in various aspects, a composite construction and is not necessarily a pure substance. A microparticle may be spherical or any other shape.

Formulations for microparticles as described herein are prepared, in various aspects, using a pharmaceutically acceptable “carrier” which when administered to an individual does not cause undesirable biological side effects or unwanted interactions. In various aspects, the “carrier” is any and every component present in the pharmaceutical formulation other than the active ingredient or ingredients. The term “carrier” includes, but is not limited to, solvents, suspending agents, dispersants, buffers, pH modifying agents, isotonicity modifying agents, preservatives, antimicrobial agents, and combinations thereof.

“Poorly water soluble active agent” as used herein refers to an active agent having a solubility of less than 10 mg/ml at 25° C., less than 5 mg/ml at 25° C., less than 1 mg/ml at 25° C., or less than 0.5 mg/ml at 25° C.

“Water-soluble active agent” as used herein refers to an active agent having a solubility of greater than 10 mg/ml at 25° C., greater than 25 mg/ml at 25° C., greater than 50 mg/ml at 25° C., or greater than 100 mg/ml at 25° C.

“Hydrophilic polymer” as used herein refers to a polymer that has an affinity for water, though is not necessarily water soluble.

“Hydrophobic polymer”, as used herein, refers to polymers that tend to repel water.

### II. COMPOSITIONS

### A. Active Agents

The microparticle compositions described herein contain one or more active agents. The active agent is either water soluble, poorly water soluble, or water insoluble. In various embodiments, the active agent is useful for treating diseases or disorders of the eye. Suitable classes of active agents include, but are not limited to, active agents that lower intraocular pressure, antibiotics, anti-inflammatory agents, chemotherapeutic agents, and steroids. The active agents described herein are administered alone or in combination with other active agents to treat diseases or disorders of the eyes. In other words, a microparticle is contemplated containing more than one active agent.

In various aspects, a poorly water soluble active agent can be co-administered with a water-soluble active agent, either in the same microparticle or in different microparticle or microparticles. A water-soluble active agent is, in various aspects, formulated in polymeric microparticles in which the hydrophilicity, molecular weight, and/or monomer composition has been optimized to maximize loading of the active agent in the microparticles.

Microparticles containing water-soluble active agents, and methods of making and using thereof, are described herein and in WO 2008/157614.

1. Active Agents that Lower IOP

In various embodiments, the microparticles contain an active agent that reduces elevated IOP in the eye. Suitable active agents include, but are not limited to, prostaglandins analogs, such as travoprost, bimatoprost, latanoprost, unoprostine, and combinations thereof; and carbonic anhydrase inhibitors (CAI), such as methazolamide, and 5-acylimino- and related imino-substituted analogs of methazolamide; and combinations thereof.

### 2. Antibiotics

Microparticles or compositions comprising a microparticle are contemplated that contain one or more poorly water soluble antibiotics. Exemplary antibiotics include, but are not limited to, cephaloridine, cefamandole, cefamandole nafate, cefazolin, cefoxitin, cephacetrile sodium, cephalexin, cephaloglycin, cephalosporin C, cephalothin, cafcillin, cephamycins, cephapirin sodium, cephradine, penicillin BT, penicillin N, penicillin O, phenethicillin potassium, pivampiculin, amoxicillin, ampicillin, cefatoxin, cefotaxime, moxalactam, cefoperazone, cefsulodin, ceflizoxime, ceforanide, cefiaxone, ceftazidime thienamycin, N-formimidoyl thienamycin, clavulanic acid, penemcarboxylic acid, piperacillin, sulbactam, cyclosporins, and combinations thereof.

### 3. Inhibitors of Growth Factor Receptors

In another embodiment, the poorly water soluble active agent is an inhibitor of a growth factor receptor. Suitable inhibitors include, but are not limited to, inhibitors of Epidermal Growth Factor Receptor (EGFR), such as AGI 478, and EGFR kinase inhibitors, such as BIBW 2992, erlotinib, gefitinib, lapatinib, and vandetanib.

AG 1478 is a potent inhibitor of the epidermal growth factor receptor (EGFR). It was developed initially as a small-molecule tyrosine kinase antagonist to treat tumors, such as breast and ovarian, that have large excesses of EGFR on their surfaces. EGFR is present in many cell types in the body and is responsible for mediating basic cell behaviors such as proliferation and fate choice of cells, thus making systemic knockdown of EGFR problematic.

### 4. Chemotherapeutic Agents and Steroids

Microparticle or microparticle compositions are provided that contain one or more poorly water soluble chemotherapeutic agents and/or steroids. In various embodiments, the poorly water soluble chemotherapeutic agent is methotrexate. Methotrexate is an antimetabolite which has been used to treat autoimmune disorders as well as certain types of cancers. In the eye, methotrexate is used to treat a number of inflammatory diseases, such as uveitis. Methotrexate is known to cause adverse side effects when administered systemically. Sustained, local delivery has the potential to reduce the amount of methotrexate in serum or eliminate it completely and thus mitigate adverse side effects.

In various embodiments, the agent is a poorly water soluble steroid, such as, and without limitation, prednisolone acetate, triamcinolone, prednisolone, hydrocortisone, hydrocortisone acetate, hydrocortisone valerate, vidarabine, fluorometholone, fluocinolone acetonide, triamcinolone acetonide, dexamethasone, dexamethasone acetate, and combinations thereof.

### 5. Pharmaceutically Acceptable Salts

The active agent, in various embodiments, is administered as the free acid or base or as a pharmaceutically acceptable acid addition or base addition salt.

Examples of pharmaceutically acceptable salts include, but are not limited to, mineral or organic acid salts of basic residues such as amines; and alkali or organic salts of acidic residues such as carboxylic acids. The pharmaceutically acceptable salts include the conventional non-toxic salts or the quaternary ammonium salts of the parent compound formed, for example, from non-toxic inorganic or organic acids. Such conventional nontoxic salts include those derived from inorganic acids such as hydrochloric, hydrobromic, sulfuric, sulfamic, phosphoric, and nitric acids; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, tolunesulfonic, naphthalenesulfonic, methanesulfonic, ethane disulfonic, oxalic, and isethionic salts.

The pharmaceutically acceptable salts of the compounds is, in various aspects, synthesized from the parent compound, which contains a basic or acidic moiety, by conventional chemical methods. Generally, such salts are, in various aspects, prepared by reacting the free acid or base forms of these compounds with a stoichiometric amount of the appropriate base or acid in water or in an organic solvent, or in a mixture of the two; generally, non-aqueous media like ether, ethyl acetate, ethanol, isopropanol, or acetonitrile are preferred. Lists of suitable salts are found in Remington's Pharmaceutical Sciences, 20th ed., Lippincott Williams & Wilkins, Baltimore, Md., 2000, p. 704; and “Handbook of Pharmaceutical Salts: Properties, Selection, and Use,” P. Heinrich Stahl and Camille G. Wermuth, Eds., Wiley-VCH, Weinheim, 2002.

As generally used herein “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

### B. Polymers

The microparticles described here are, in various aspects, formed from natural and/or synthetic polymeric materials wherein poly(D,L-lactic acid) is combined with a second polymer. The second “polymer” or “polymeric,” in various embodiments is contemplated to include oligomers, adducts, random copolymers, pseudo-copolymers, statistical copolymers, alternating copolymers, periodic copolymer, bipolymers, terpolymers, quaterpolymers, other forms of copolymers, substituted derivatives thereof, and combinations of two or more thereof (i.e., polymer blends). The other polymer in various aspects is linear, branched, block, graft, monodisperse, poly disperse, regular, irregular, tactic, isotactic, syndiotactic, stereoregular, atactic, stereoblock, single-strand, double-strand, star, comb, dendritic, and/or ionomeric. Biodegradable polymers are contemplated for use in combination with poly(D,L-lactic acid), so long as they are biocompatible, and include for example, and without limitation polyhydroxyacids and polyhydroxyalkanoates.

Microparticle of the disclosure offer advantages over microparticle previously disclosed, at least in part, due to characteristic imparted by the percentage of poly(D,L-lactic acid) in the microparticle core. Previously disclosed poly(D,L-lactic acid) microparticles were shown to be advantageous for sustained active agent delivery when the percentage of poly(D,L-lactic acid) was at least 50%. Unexpectedly, results described herein demonstrate that a poly(D,L-lactic acid) percentage of less than 50% provides significant improvement over microparticles previously known in the art.

Accordingly, microparticles provided herein include at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, at least about 25%, at least about 26%, at least about 27%, at least about 28%, at least about 29%, at least about 30%, at least about 31%, at least about 32%, at least about 33%, at least about 34%, at least about 35%, at least about 36%, at least about 37%, at least about 38%, at least about 39%, at least about 40%, at least about 41%, at least about 42%, at least about 43%, at least about 44%, at least about 45%, at least about 46%, at least about 47%, at least about 48%, or at least about 49% up to less than 50% poly(D,L-lactic acid).

In various aspects, the microparticles comprise two polymer components wherein one polymer is a poly(D,L-lactic acid). As long as poly(D,L-lactic acid) is present in a percent amount as described herein, it will be appreciated that that microparticles including any number or type of other polymer as described herein are contemplated.

Polymers suitable for use with poly(D,L-lactic acid) have been described in great detail in the prior art. They include, but are not limited to: polyamides, polycarbonates, polyalkylenes, polyalkylene glycols, polyalkylene oxides, polyalkylene terephthalates, polyvinyl alcohols, polyvinyl ethers, polyvinyl esters, polyvinyl halides, polyvinylpyrrolidone, polyglycolides, polysiloxanes, polyurethanes and copolymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, poly(methyl methacrylate), poly(ethylmethacrylate), poly(butylmethacrylate), poly (isobutylmethacrylate), poly(hexy lmethacrylate), poly(isodecylmethacrylate), poly(lauryl methacrylate), poly(phenyl methacrylate), poly (methyl acrylate), poly(isopropyl aery late), poly(isobutyl acrylate), poly(octadecyl acrylate), polyethylene, polypropylene polyethylene glycol), poly(ethylene oxide), poly(ethylene terephthalate), polyvinyl alcohols), polyvinyl acetate), poly vinyl chloride polystyrene and polyvinylpyrrolidone.

In various embodiments, the microparticles are made from PLGA and poly(D,L-lactic acid) (PLA). The molecular weight of PLGA, in various aspects, is from about 10 kD to about 80 kD, from about 10 kD to about 75 kD, from about 10 kD to about 70 kD, from about 10 kD to about 65 kD, from about 10 kD to about 60 kD, from about 10 kD to about 55 kD, from about 10 kD to about 50 kD, from about 10 kD to about 45 kD, from about 10 kD to about 40 kD, or from about 10 kD to about 35 kD.

The molecular weight range of PLA, in various aspects, is from about 10 to about 50 kDa, from about 10 kD to about 55 kD, from about 10 kD to about 50 kD, from about 10 kD to about 45 kD, from about 10 kD to about 40 kD, from about 10 kD to about 30 kD, from about 10 kD to about 25 kD, from about 15 kD to about 40 kD, from about 20 kD to about 40 kD, from about 25 kD to about 40 kD, from about 30 kD to about 40 kD, or from about 35 kD to about 40 kD.

In embodiments utilizing PLGA, the ratio of lactide to glycolide is, in various aspects, from about 75:25 to about 50:50. In various embodiment, the ratio is about 50:50, about 55:45, about 60:40 about 65:35, about 70:30, or about 75:25. Exemplary PLGA polymers include, but are not limited to, poly(D,L-lactic-co-glycolic acid) (PLGA, 50:50 lactic acid to glycolic acid ratio, Mₙ=10 kDa, referred to as 502H); poly(D,L-lactic-co-glycolic acid) (PLGA, 50:50 lactic acid to glycolic acid ratio, Mₙ=25 kDa, referred to as 503H); poly(D,L-lactic-co-glycolic acid) (PLGA, 50:50 lactic acid to glycolic acid ratio, Mₙ=30 kDa, referred to as 504H); poly(D,L4actic-co-glycolic acid) (PLGA₅ 50:50 lactic acid to glycolic acid ratio, Mₙ=35 kDa, referred to as 504); and poly(D,L-lactic-co-glycolic acid) (PLGA, 75:25 lactic acid to glycolic acid ratio, Mₙ=10 kDa, referred to as 752). The designation “H” means the polymer is terminated with a carboxylic acid group. When combined with PLA, the PLGA containing microparticles are referred to as PLGA:PL.

### C. Percent Loading

The percent loading of a microparticles is modulated by “matching” the hydrophilicity or hydrophobicity of the polymer to the agent to be encapsulated. When utilizing PLGA, this match can be achieved by selecting the monomer ratios so that the copolymer is more hydrophilic for hydrophilic active agents or less hydrophilic for hydrophobic active agents. Alternatively, the polymer can be made more hydrophilic, for example, by introducing carboxyl groups onto the polymer. Combinations of a hydrophilic active agent and a hydrophobic active agent are contemplated when the microparticle is prepared from a hydrophilic PLGA and a hydrophobic PLA polymer.

The percent loading of the active agent in the microparticles is, in various aspects, from about 1 to 50 weight percent, 5 to 30 weight percent, or 10 to 20 weight percent.

### D. Release Kinetics

The microparticle compositions described herein release an effective amount the active agent to a degree suitable for treating the target indication. In various aspects, the active agent is released for a period greater than 14 days in vivo, greater than 60 days in vivo, greater than 75 days in vivo, greater than 90 days in vivo, greater than 100 days in vivo, greater than 120 days in vivo, greater than 150 days in vivo, greater than 200 days in vivo or, greater than 250 days in vivo.

Previously it was shown that, with respect to AGI 478, release was greater for PLGA 503H microparticles prepared using an oil-in-water emulsion technique having an active agent loading of 5.0% compared to a loading of 2.5%. The microparticles exhibited a more rapid release of drag over the first 50 days, followed by a more linear release over the next 125 days. Hydrophilicity of the polymer influences the release profile of AG 1478. For example, release of AG 1478 was greater from microparticles prepared from PLGA having carboxylic end groups, such as PLGA 503H and 504H, compared to the non-carboxylated polymer, PLGA 504, using the oil-in-water cosolvent technique. The microparticles provided herein comprising PLA and, for example, PLGA provide an improvement in terms of release kinetics, over these previously disclosed microparticles.

### E. Preparation of Microparticles

### 1. Solvents

Typical solvents are organic solvents such as methylene chloride, which leave low levels of residue that are generally accepted as safe. Suitable water-insoluble solvents include methylene chloride, chloroform, carbon tetrachloride, dicholorethane, ethyl acetate and cyclohexane. Additional solvents include, but are not limited to, alcohols such as methanol (methyl alcohol), ethanol, (ethyl alcohol), 1-propanol (n-propyl alcohol), 2-propanol (isopropyl alcohol), 1-butanol (n-butyl alcohol), 2-butanol (sec-butyl alcohol), 2-methyl-1-propanol (isobutyl alcohol), 2-methyl-2-propanol (t-butyl alcohol), 1-pentanol (n-pentyl alcohol), 3-methyl-1-butanol (isopentyl alcohol), 2,2-dimethyl-1-propanol (neopentyl alcohol), cyclopentanol (cyclopentyl alcohol), 1-hexanol (n-hexanol), cyclohexanol (cyclohexyl alcohol), 1-heptanol (n-heptyl alcohol), 1-octanol (n-octyl alcohol), 1-nonanol (n-nonyl alcohol), 1-decanol (n-decyl alcohol), 2-propen-1-ol (allyl alcohol), phenylmethanol (benzyl alcohol), diphenylmethanol (diphenylcarbinol), triphenylmethanol (triphenylcarbinol), glycerin, phenol, 2-methoxy ethanol, 2-ethoxyethanol, 3-ethoxy-1,2-propanediol, Di(ethylene glycol) methyl ether, 1,2-propanediol, 1,3-propanediol, 1,3-butanediol, 2,3-butanediol, 1,4-butanediol, 1,2-pentanediol, 1,3-pentanediol, 1,4-pentanediol, 1,5-pentanediol, 2,3-pentanediol, 2,4-pentanediol, 2,5-pentanediol, 3,4-pentanediol, 3,5-pentanediol, and combinations thereof.

### 2. Excipients for Administration to the Eye

Considerations in the formulation of the microparticle compositions include, but are not limited to, sterility, preservation, isotonicity, and buffering. The preparation of ophthalmic solutions and suspensions are described in Ansel, et al, Pharmaceutical Dosage Forms and Active agent Delivery Systems 6^{th} Ed, pp. 396-408, Williams and Wilkins (1995). Suspensions are often more advantageous than solution as they typically have increased corneal contact time and thus can provide higher efficacy. Ophthalmic suspensions must contain particles of appropriate chemical characteristics and size to be non-irritating to the eyes. The suspension must also not agglomerate upon administration. Excipients, such as dispersants, can be included to prevent aggregation of the particles.

The microparticles are typically suspended in sterile saline, phosphate buffered saline, or other pharmaceutically acceptable carriers for administration to the eye.

Materials that used to formulate or prepare the microparticles include without limitation anionic, cationic, amphoteric, and non-ionic surfactants. Anionic surfactants include without limitation di-(2 ethylhexyl) sodium sulfosuccinate; non-ionic surfactants include the fatty acids and the esters thereof; surfactants in the amphoteric group include (1) substances classified as simple, conjugated and derived proteins such as the albumins, gelatins, and glycoproteins, and (2) substances contained within the phospholipid classification, for example, lecithin. The amine salts and the quaternary ammonium salts within the cationic group also comprise useful surfactants. Other surfactant compounds useful to form coacervates include polysaccharides and their derivatives, the mucopolysaccharides and the polysorbates and their derivatives. Synthetic polymers that may be used as surfactants include compositions such as polyethylene glycol and polypropylene glycol. Further examples of suitable compounds that may be utilized to prepare coacervate systems include glycoproteins, glycolipids, galactose, gelatins, modified fluid gelatins and galacturonic acid.

Hydrophobic surfactants such as, and without limitation, fatty acids and cholesterol are added during processes to improve the resulting distribution of hydrophobic active agents in hydrophobic polymeric microparticles. Examples of fatty acids include butyric acid, valeric acid, caproic acid, enanthic acid, caprylic acid, pelargonic acid, caprylic acid, undecylic acid, lauric acid, tridecylic acid, myristic acid, pentadecylic acid, palmitic acid, heptadecylic acid, stearic acid, nonadecanoic acid, arachic acid, isocrotonic acid, undecylenic acid, oleic acid, elaidic acid, sorbic acid, linoleic acid, linolenic acid and arachidonic acid.

### 3. Methods of Making

In various embodiments, the surfactant polyvinyl alcohol is used to prepare the microparticles. Studies indicate that percent loading is dependent on the nature of surfactant used in the double emulsion methods described above. For example, using the PLGA/PLA blend and 20% timolol maleate by weight, a loading of 18.76 μg of timolol/mg of spheres was obtained when a 5% PVA solution was used. In contrast, the load of timolol was 2.3 μg per mg of spheres when the spheres were prepared using a 5% poly(ethylene˜alt-maleic anhydride) (PEMA) solution. HI. Methods of Making

There are several processes whereby microparticles can be made, including, but not limited to, spray drying, interfacial polymerization, hot melt encapsulation, phase separation encapsulation, spontaneous emulsion, solvent evaporation microencapsulation, solvent removal microencapsulation, coacervation, low temperature microparticle formation, and phase inversion nanoencapsulation (“PtM”).

The dispersion of the one or more active agents within the polymer matrix can be enhanced by varying: (1) the solvent used to solvate the polymer; (2) the ratio of the polymer to the solvent; (3) the particle size of the material to be encapsulated; (4) the percentage of the active agent(s) relative to the polymer (e.g., active agent loading); and/or the polymer concentration.

The following are representative methods for forming microparticles.

a. Spray Drying

In spray drying, the core material to be encapsulated is dispersed or dissolved in a solution. Typically, the solution is aqueous and preferably the solution includes a polymer. The solution or dispersion is pumped through a micronizing nozzle driven by a flow of compressed gas, and the resulting aerosol is suspended in a heated cyclone of air, allowing the solvent to evaporate from the microdroplets. The solidified microparticles pass into a second chamber and are trapped in a collection flask. Interfacial POIY condensation

Interfacial polycondensation is used to microencapsulate a core material in the following manner. One monomer and the core material are dissolved in a solvent. A second monomer is dissolved in a second solvent (typically aqueous) which is immiscible with the first. An emulsion is formed by suspending the first solution through stirring in the second solution. Once the emulsion is stabilized, an initiator is added to the aqueous phase causing interfacial polymerization at the interface of each droplet of emulsion.

b. Hot Melt Encapsulation

In hot melt microencapsulation, the core material (to be encapsulated) is added to molten polymer. This mixture is suspended as molten droplets in a nonsolvent for the polymer (often oil-based) which has been heated to approximately 10° C. above the melting point of the polymer. The emulsion is maintained through vigorous stirring while the nonsolvent bath is quickly cooled below the glass transition of the polymer, causing the molten droplets to solidify and entrap the core material.

c. Solvent Evaporation Microencapsulation

In solvent evaporation microencapsulation, the polymer is typically dissolved in a water immiscible organic solvent and the material to be encapsulated is added to the polymer solution as a suspension or solution in an organic solvent. An emulsion is formed by adding this suspension or solution to a beaker of vigorously stirring water (often containing a surface active agent, for example, polyethylene glycol or polyvinyl alcohol, to stabilize the emulsion). The organic solvent is evaporated while continuing to stir. Evaporation results in precipitation of the polymer, forming solid microcapsules containing core material.

The solvent evaporation process can be used to entrap a liquid core material in a polymer such as PLA-containing copolymer. The copolymer is dissolved in a miscible mixture of solvent and nonsolvent, at a nonsolvent concentration which is immediately below the concentration which would produce phase separation (i.e., cloud point). The liquid core material is added to the solution while agitating to form an emulsion and disperse the material as droplets. Solvent and nonsolvent are vaporized, with the solvent being vaporized at a faster rate, causing the polymer or copolymer to phase separate and migrate towards the surface of the core material droplets. This phase-separated solution is then transferred into an agitated volume of nonsolvent, causing any remaining dissolved polymer or copolymer to precipitate and extracting any residual solvent from the formed membrane. The result is a microcapsule composed of polymer or copolymer shell with a core of liquid material.

Solvent evaporation microencapsulation can result in the stabilization of insoluble or poorly soluble active agent particles in a polymeric solution for a period of time ranging from 0.5 hours to several months.

d. Stabilization

The stabilization of insoluble or poorly soluble active agent particles within the polymeric solution could be critical during scale-up. By stabilizing suspended particles within the dispersed phase, said particles can remain homogeneously dispersed throughout the polymeric solution as well as the resulting polymer matrix that forms during the process of microencapsulation. The homogeneous distribution of active agent particles can be achieved in any kind of device, including microparticles, rods, films, and other device. Solvent evaporation microencapsulation (SEM) has several advantages. SEM allows for the determination of the best polymer-solvent-insoluble particle mixture that will aid in the formation of a homogeneous suspension that can be used to encapsulate the particles. SEM stabilizes the insoluble particles or within the polymeric solution, which will help during scale-up because one will be able to let suspensions of insoluble particles sit for long periods of time, making the process less time-dependent and less labor intensive. SEM allows for the encapsulated particles to remain suspended within a polymeric solution for up to 30 days, which may increase the amount of insoluble material entrapped within the polymeric matrix, potentially improving the physical properties of the active agent delivery vehicle. SEM allows for the creation of microparticles that have a more optimized release of the encapsulated material. For example, if the insoluble particle is localized to the surface of the microparticle, the system will have a large ‘burst’ effect. In contrast, creating a homogeneous dispersion of the insoluble particle within the polymeric matrix will help to create a system with release kinetics that begin to approach the classical ‘zero-ordered’ release kinetics that are often perceived as being ideal in the field of active agent delivery).

e. Oil-In-Water Emulsion Cosolvent Technique

In various embodiments embodiment, the microparticles are prepared using an oil-in-water emulsion co-solvent technique, in which an organic co-solvent, such as DMSO₅ is used to prepare the microparticles.

f. Solvent Removal Microencapsulation

In solvent removal microencapsulation, the polymer is typically dissolved in an oil miscible organic solvent and the material to be encapsulated is added to the polymer solution as a suspension or solution in organic solvent. Surface active agents can be added to improve the dispersion of the material to be encapsulated. An emulsion is formed by adding this suspension or solution to vigorously stirring oil, in which the oil is a nonsolvent for the polymer and the polymer/solvent solution is immiscible in the oil. The organic solvent is removed by diffusion into the oil phase while continuing to stir. Solvent removal results in precipitation of the polymer, forming solid microcapsules containing core material.

g. Phase Separation Microencapsulation

In phase separation microencapsulation, the material to be encapsulated is dispersed in a polymer solution with stirring. While continually stiffing to uniformly suspend the material, a nonsolvent for the polymer is slowly added to the solution to decrease the polymer's solubility. Depending on the solubility of the polymer in the solvent and nonsolvent, the polymer either precipitates or phase separates into a polymer rich and a polymer poor phase. Under proper conditions, the polymer in the polymer rich phase will migrate to the interface with the continuous phase, encapsulating the core material in a droplet with an outer polymer shell.

h. Spontaneous Emulsification

Spontaneous emulsification involves solidifying emulsified liquid polymer droplets by changing temperature, evaporating solvent, or adding chemical cross-linking agents. The physical and chemical properties of the encapsulant, and the material to be encapsulated, dictates the suitable methods of encapsulation. Factors such as hydrophobicity, molecular weight, chemical stability, and thermal stability affect encapsulation.

i. Coacervation

Encapsulation procedures for various substances using coacervation techniques have been described in the prior art, for example, in GB-B-929 406; GB-B-929 401; U.S. Pat. Nos. 3,266,987; 4,794,000 and 4,460,563. Coacervation is a process involving separation of colloidal solutions into two or more immiscible liquid layers (Ref. Dowben, R. General Physiology, Harper & Row, New York, 1969, pp. 142-143.). Through the process of coacervation compositions comprised of two or more phases and known as coacervates may be produced. The ingredients that comprise the two phase coacervate system are present in both phases; however, the colloid rich phase has a greater concentration of the components than the colloid poor phase.

j. Phase Inversion Nanoencapsulation (“PIN”)

Another process is PIN. In PIN, a polymer is dissolved in an effective amount of a solvent. The agent to be encapsulated is also dissolved or dispersed in the effective amount of the solvent. The polymer, the agent and the solvent together form a mixture having a continuous phase, wherein the solvent is the continuous phase. The mixture is introduced into an effective amount of a nonsolvent to cause the spontaneous formation of the microencapsulated product, wherein the solvent and the nonsolvent are miscible. PIN has been described by Mathiowitz et al., in U.S. Pat. Nos. 6,131,211 and 6,235,224. A hydrophobic agent is dissolved in an effective amount of a first solvent that is free of polymer. The hydrophobic agent and the solvent form a mixture having a continuous phase. A second solvent and then an aqueous solution are introduced into the mixture. The introduction of the aqueous solution causes precipitation of the hydrophobic agent and produces a composition of micronized hydrophobic agent having an average particle size of 1 micron or less.

k. Other Techniques

Other preparative processes include those that use a mixed solvent including at least one water-insoluble solvent and water that contains a surfactant, such as PVA. The active agent is either dissolved or dispersed together with a substance that has a high molecular weight (such as a polymer) into an organic solvent composition, optionally containing non-ionic surfactants of various hydrophilic-lipopliilic ratios. The composition is then introduced into an aqueous solution that contains a surfactant like PVA. The water-insoluble solvent forms an oil phase (inner phase) and is stirred into the aqueous solution as a water phase (outer phase). The O/W emulsion is combined with fresh water that contains surfactant such as PVA and is stirred to help aid the solvent evaporation. The aqueous solution contains an activator such as polyvinyl alcohol, whereby the oil phase is enclosed as small droplets within the aqueous solution as shells. The proportion of the water-miscible solvent in the oil phase is from 5% to 95%. An important aspect of this improved method is the use of high shear during the initial mixing phase, which is achievable, for example, using sonication for a period of one hour, with stirring, to uniformly mix in high amounts of active agent particles in the polymer liquefied by dissolution or by melting.

### 1. Melt-Solvent Evaporation Method

In the melt-solvent evaporation method, the polymer is heated to a point of sufficient fluidity to allow ease of manipulation (for example, stiffing with a spatula). The temperature required to do this is dependent on the intrinsic properties of the polymer. For example, for crystalline polymers, the temperature will be above the melting point of the polymer. After reaching the desired temperature, the agent is added to the molten polymer and physically mixed while maintaining the temperature. The molten polymer and agent are mixed until the mixture reaches the maximum level of homogeneity for that particular system. The mixture is allowed to cool to room temperature and harden. This may result in melting of the agent in the polymer and/or dispersion of the agent in the polymer. This can result in an increase in solubility of the active agent when the mixture is dissolved in organic solvent. The process is easy to scale up since it occurs prior to encapsulation. High shear turbines may be used to stir the dispersion, complemented by gradual addition of the agent into the polymer solution until the desired high loading is achieved. Alternatively the density of the polymer solution may be adjusted to prevent agent settling during stirring.

This method increases microparticle loading as well as uniformity of the resulting microparticles and of the agent within the microparticles. When an agent is formed into microparticles by double-emulsion solvent evaporation, transfer of the agent from the inner phase to the outer water phase can be prevented. This makes it possible to increase the percentage of agent entrapped within the microparticles, resulting in an increased amount of the active agent in the microparticles.

### 4. Agent Loading and Distribution

The distribution of the agent in particles can also be made more uniform. This uniformity can improve the release kinetics of the agent. Generally, the agent is dissolved or dispersed together with a substance that has a high molecular weight in an organic solvent composition; with or without non-ionic surfactants of various hydrophilic-lipophilic ratios. The composition is introduced into an aqueous solution that contains a surfactant like PVA. The water-insoluble solvent forms an oil phase (inner phase) and is stirred into the aqueous solution as a water phase (outer phase). The O/W emulsion is combined with fresh water that contains PVA and is stirred to help aid the solvent evaporation. The aqueous solution contains an activator such as polyvinyl alcohol, whereby the oil phase is enclosed as small droplets within the aqueous solution as shells.

In various embodiments, the microparticles are formed using a water-in-oil double emulsion (w/o/w) solvent evaporation technique. For example, the one or more active agents are dissolved in deionized water. The polymer is dissolved in an organic solvent or cosolvent. The aqueous and organic phases are emulsified via vortexing to obtain the desired active agent to polymer ratio (e.g., 10%, 20%, or greater). The emulsion is then added dropwise to an aqueous solution of a surfactant (such as polyvinyl alcohol) and allowed to stir/harden for 3 hours. The resulting microparticles are collected, such as by centrifugation, washed with deionized water, and dried (e.g., freeze drying). As demonstrated by the examples, the percent loading is increased by “matching” the hydrophilicity or hydrophobicity of the polymer to the agent to be encapsulated. In some cases, such as PLGA, this can be achieved by selecting the monomer ratios so that the copolymer is more hydrophilic. Alternatively, the polymer is made more hydrophilic, for example, by treating the polymer with a carboxyl solution.

The percent loading the active agent in the microparticles is from about 1 to about 50 wt %, preferably from about 1 to about 20 wt %, more preferably from about 10 to about 20 wt %. In one embodiment, the percent loading of the active agent is 10 wt % or 20 wt %. As discussed herein, the percent loading is increased by “matching” the hydrophilicity or hydrophobicity of the polymer to the poorly water soluble agent to be encapsulated. Alternatively, the polymer is made more hydrophilic, for example, by introducing carboxyl groups on to the polymer. A combination of a hydrophilic active agent and a hydrophobic active agent is, in various aspects, encapsulated in microparticles prepared from a blend of a more hydrophilic PLGA and a hydrophobic polymer, such as PLA.

### III. METHODS OF USE

The microparticle compositions described herein are in various aspects administered to treat or prevent diseases or disorders. The dosage of the active agent which is released at the site of administration should be bioequivalent as defined by the Food and Active agent Administration for the active agent when administered in solution, suspension or enterally, in the absence of the microparticles.

### A. Glaucoma

In various embodiment, the microparticle compositions are administered to manage (e.g., reduce) IOP in patients needing such treatment, for example, patients suffering from glaucoma. Glaucoma is an ophthalmic disease characterized by the gradual degeneration of retinal ganglion cells (RGCs). RGCs synapse with bipolar cells and transmit visual inputs to the brain along the optic nerve. Degeneration of these cells leads to gradual vision loss and ultimately blindness if untreated.

### B. Neural Regeneration

The microparticles described herein are used to deliver one or more active agents that promote neural regeneration, for example, in patients suffering from glaucoma. AG1 478 has been shown to promote neural regeneration. AG 1478 is an inhibitor of EGFR.

The neural degeneration in glaucoma is accompanied by extensive remodeling of the extracellular matrix (ECM) including the production of chondroitin sulfate proteoglycans (CSPGs) which inhibit regeneration. Administration of an EGFR inhibitor, such as AG 1478, has been shown to lead to a reduction in activated astrocytes, a reduction in the production of CSPGs, and regeneration in the optic nerve.

AG 1478 is also, in various embodiments, co-administered with neural progenitor cells to replace lost retinal ganglion cells (RGCs) along with sustained delivery of AG1 478 to promote regeneration. The optic nerve crush model is an excellent first model for studying methods to promote regeneration in glaucoma as well as in the CNS more broadly.

Recent work suggests that the EGFR plays an important role in regulating the production of CSPGs and maintaining specific astrocyte phenotype. EGFR, also known as human EGF receptor (HER) and ErbBl, is a member of a family of transmembrane proteins with tyrosine kinase activity. EGFR has seven different but structurally similar ligands, including EGF₅ transforming growth factor-β1 (TGF-P1), and transforming growth factor-α (TGF-α). EGFR activation controls cell migration, apoptosis, protein secretion and differentiation. Activation of EGFR has been shown to affect the behavior of astrocytes. Ligands of EGFR stimulate astrocyte proliferation and differentiation, induce morphological changes and process formation, and enhance their mobility in vitro. In glaucomatous optic neuropathy, EGFR activation is increased in astrocytes and their activation in the cribriform plates to the damaged optic nerve bundles creates compression, backward bowing, and disorganization of the optic nerve head-characteristic features of glaucomatous eyes with high or normal intraocular pressure.

The EGFR ligands EGF and TGF-β1 greatly increase CSPG production after injury, including neurocan and phosphacan, while upregulation of CSPGs by astrocytes is mediated specifically by the EGFR receptor. In addition, activation of EGFR causes optic nerve astrocytes and brain astrocytes to form cribriform structures with cavernous spaces, similar to the structures that reactive astrocytes form in the glial scar. EGFR also plays a role in astrocyte phenotype. In normal tissue astrocytes are quiescent, producing only a moderate amount of CSPGs and retaining a stellate morphology. After injury, these quiescent astrocytes are activated and become reactive, with elongated processes and increased motility.

Astrocytes upregulate and activate EGFR in three different optic nerve injury models: transient eye ischemia, chronic glaucoma, and optic nerve transection. However, application of a commercially available EGFR tyrosine kinase inhibitor, AG1478—a potent, reversible antagonist of EGFR—in a rodent model of glaucomatous optic neuropathy and an optic nerve crush model, reverses this upregulation and activation of astrocytes and increases the survival of RGCs. Further, evidence shows that EGFR activation mediates inhibition of axon regeneration in retinal explants by production of CSPGs and myelin. These studies provide evidence for the idea that modulating the behavior of astrocytes via EGFR signaling is an attractive candidate for treatment of CNS disorders.

### C. Uveitis

Uveitis specifically refers to inflammation of the middle layer of the eye, termed the “uvea” but in common usage may refer to any inflammatory process involving the interior of the eye. Uveitis is estimated to be responsible for approximately 10% of the blindness in the United States. Uveitis requires an urgent referral and thorough examination by an ophthalmologist, along with urgent treatment to control the inflammation. Uveitis is usually categorized anatomically into anterior, intermediate, posterior and panuveitic forms. Anywhere from two-thirds to 90% of uveitis cases are anterior in location (anterior uveitis), frequently termed iritis—or inflammation of the iris and anterior chamber. This condition can occur as a single episode and subside with proper treatment or may take on a recurrent or chronic nature. Symptoms include red eye, injected conjunctiva, pain and decreased vision. Signs include dilated ciliary vessels, presence of cells and flare in the anterior chamber, and keratic precipitates (“KP”) on the posterior surface of the cornea. Intermediate uveitis consists of vitritis—inflammatory cells in the vitreous cavity, sometimes with snowbanking, or deposition of inflammatory material on the pars plana. Posterior uveitis is the inflammation of the retina and choroid. Pan-uveitis is the inflammation of all the layers of the uvea.

A myriad of conditions can lead to the development of uveitis, including systemic diseases as well as syndromes confined to the eye. In anterior uveitis, no specific diagnosis is made in approximately one-half of cases. However, anterior uveitis is often one of the syndromes associated with HLA-B27.

The prognosis is generally good for those who receive prompt diagnosis and treatment, but serious complication (including cataracts, glaucoma, band keratopathy, retinal edema and permanent vision loss) may result if left untreated. The type of uveitis, as well as its severity, duration, and responsiveness to treatment or any associated illnesses, all factor in to the long term prognosis. Uveitis can be treated using steroids, such as prednisolone, and chemotherapeutic agents, such as methotrexate. Thus, in various embodiments, the microparticles are loaded with ofloxacin, prednisolone, or a combination thereof.

### D. Post Surgical Ocular Inflammation/Infection

Most surgeries involving the eye are followed by ocular inflammation and/or infection. Topical administration of eye drops containing a combination of a steroid and an antibiotic is the predominant treatment for controlling inflammation as well as infection. Although such eye drops have been shown to be effective, poor compliance and the risk of re-opening of the stitched wound due to continuous touching of the wound when applying the eye drops remain fundamental issues. Therefore, it is desirable to provide a long-term ocular delivery system that provides release of the active agents for approximately 2-3 weeks in order to minimize dosing frequency, improve patient compliance, reduce side effects due to systemic absorption of the active agents, and keep the stitched wound intact. In one embodiment, microparticles loaded with an antibiotic, a steroid, or combinations thereof are administered to a patient post eye surgery. In various embodiments, the microparticles are loaded with ofloxacin, prednisolone, or a combination thereof.

### E. Dry Eye Syndrome

Dry eye syndrome (Keratoconjunctivitis sicca (KCS)) is one of the most common problems treated by eye physicians. Over ten million Americans suffer from dry eyes. It is usually caused by a problem with the quality of the tear film that lubricates the eyes.

Dry eye syndrome has many causes. One of the most common reasons for dryness is simply the normal aging process. As we grow older, bodies produce less oil—60% less at age 65 then at age 18. This is more pronounced in women, who tend to have drier skin then men. The oil deficiency also affects the tear film. Without as much oil to seal the watery layer, the tear film evaporates much faster, leaving dry areas on the cornea.

Many other factors, such as hot, dry or windy climates, high altitudes, air-conditioning and cigarette smoke also cause dry eyes. Contact lens wearers may also suffer from dryness because the contacts absorb the tear film, causing proteins to form on the surface of the lens. Certain medications, thyroid conditions, vitamin A deficiency, and diseases such as Parkinson's and Sjogren's can also cause dryness.

Inflammation occurring in response to tears film hypertonicity can be treated by administering the microparticles described herein loaded with poorly water soluble steroids and/or with poorly water soluble immunosuppressants.

### F. Macular Degeneration

Macular degeneration is a medical condition predominantly found in elderly adults in which the center of the inner lining of the eye, known as the macula area of the retina, suffers thinning, atrophy, and in some cases, bleeding. This can result in loss of central vision, which entails inability to see fine details, to read, or to recognize faces. According to the American Academy of Ophthalmology, it is the leading cause of central vision loss (blindness) in the United States today for those over the age of fifty years. Although some macular dystrophies that affect younger individuals are sometimes referred to as macular degeneration, the term generally refers to age-related macular degeneration (AMD or ARMD). Macular degeneration can be treated using anti-angio genesis inhibitors. In one embodiment, the microparticles are loaded with a poorly water soluble anti-angiogenesis inhibitor or growth factor for the treatment of macular degeneration.

### IV. METHODS OF ADMINISTRATION

The composition is, in various aspects, administered using a variety of techniques well known in the art including, but not limited to, topically and by injection. Suitable dosage forms include but are not limited to, ointments and solutions and suspensions, such as eye drops. In one embodiment, the compositions are administered to the eye by injection. In various embodiments, the microparticle composition is administered subconjunctivally.

“Subconjunctival” or “subconjunctivally”, as used herein, refers to administration under the conjunctiva of the eye. The conjunctiva is the clear membrane that coats the inner aspect of the eyelids and the outer surface of the eye. The microparticle compositions are generally administered as suspensions in a pharmaceutically acceptable carrier, such as phosphate buffered saline (PBS). Subconjunctival administration of active agents, typically by injection, has shown minimal concentration of active agent in the plasma and notable concentrations in the eye, including the aqueous humor.

### V. KITS

Kits are provided which contain the microparticle compositions and optionally one or more pharmaceutically acceptable excipients or carriers. In various embodiments, the kit contains the microparticles in dry powder form in one container, such as a vial, jar, or ampule, and the pharmaceutically acceptable carrier in another container, such as a vial, jar, or ampule. The kit in various aspects contains instructions for resuspending the microparticles in the carrier and for administering the composition. If excipients are present, they optionally are in one or both containers. In various embodiments, the kit contains the microparticles resuspended in the carrier and optionally one or more pharmaceutically excipients. The kit would typically contain instructions for administering the composition. The kit can also contain one or more apparatus for preparing and/or administering the compositions, such as a needle and syringe. The container(s) containing the microparticles and the carrier can be packaged using techniques well known in the art. Suitable package materials include, but are not limited to, boxes

Unless defined otherwise, all technical and scientific terms used herein have the same meanings as commonly understood by one of skill in the art to which the disclosed invention belongs. Additional aspects and details of the invention will be apparent from the following examples, which are intended to be illustrative rather than limiting.

### VI. EXAMPLES

For the example below, materials were obtained as follows.

Poly(D,L-lactic-co-glycolic acid (PLGA, 50:50 lactic acid to glycolic acid ratio, Mₙ=10 kDa, referred to as 502H); poly(D,L-lactic-co-gly colic acid (PLGA, 50:50 lactic acid to glycolic acid ratio, Mₙ=25 kDa, referred to as 503H); poly(D,L-lactic-co-glycolic acid (PLGA, 50:50 lactic acid to glycolic acid ratio, Mₙ−30 kDa, referred to as 504H); and poly(D,L-lactic-co-gly colic acid (PLGA, 50:50 lactic acid to glycolic acid ratio, Mₙ=35 kDa, referred to as 504) were purchased from Boehringer Ingelheim (Ingelheim, Germany). Poly(D,L-lactic acid) (PLA, Mₙ−˜20-30 kDa) and polyvinyl alcohol) (PVA, 88 mo1 % hydrolyzed) were purchased from Polyscienes (Warrington, Pa., USA).

Methotrexate and Prednisolone acetate were purchased from Sigma (St. Louis, Mo., USA).

All other chemicals were A.C.S. reagent grade from Sigma (St. Louis, Mo., USA).

### Example 1

### Methotrexate/Triamcinolone Microparticle Preparation

Microspheres were prepared by phase separation using the single emulsion solvent evaporation method. Two hundred milligrams of the specific polymer was dissolved in 1 mL of dicholoromethane (DCM) and 4 mL of trifluoroethanol (TFE). Then, 40 mg of ofloxacin or 40 mg of prednisolone acetate or 20 mg of methotrexate was added to this solution, and vortexed to obtain a desired active agent to polymer ratio of 20% (40 mg active agent/200 mg polymer) in case of ofloxacin, and 10% (20 mg active agent/200 mg polymer) in case of methotrexate. The organic phase was added dropwise to 200 mL of 5% (w/v) PVA aqueous solution. The aqueous and organic phases were emulsified via stirring/hardening for 3 hours. Microspheres were then collected by centrifugation, washed three times with deionized water, and freeze dried for 3 days. Blank microspheres were made at the same time under identical conditions except that no ofloxacin or methotrexate was added.

### Example 2

### In Vitro Release Study

In 1.5 mL eppendorf tubes, 10 mg of ofloxacin or prednisolone acetate or methotrexate containing microspheres or blank microspheres were suspended with 1 mL of phosphate buffered saline. Samples were prepared in triplicate. Mixtures were then incubated at 37° C. on a labquake rotating shaker (Barnstead/Thermolyne; Dubuque, Iowa USA). At specific time points (1, 3, 5, and 8 hours and 1, 3, 7 days, and once every 7 days thereafter until no pellets were present) the mixture was centrifuged and the supernatant was collected. One milliliter of phosphate buffered saline was then added to replace the withdrawn supernatant and the microspheres were resuspended and returned to the shaker. Supernatants for each of the sets of microspheres was frozen and stored at −80° C. for subsequent analysis using UV spectroscopy at 288 nm, 245 nm, and 303 nm for ofloaxacin, prednisolone acetate, and methotrexate respectively [1, 2, 3]. Concentration of dissolved ofloxacin or prednisolone acetate or methotrexate was determined as a function of time from their respective standard curves. Plotting ofloxacin or prednisolone acetate or methotrexate concentration versus UV absorbance produced a calibration curve for quantification of ofloxacin or prednisolone acetate or methotrexate. A linear fit was established from ˜0.09-25 μg/mL of ofloxacin (Y=15.196x+0.0571; r²=0.9999) or prednisolone acetate (Y=26.798x+0.1851; r²=0.9997) or methotrexate (Y=19.763x−0.0372; r²=1) in phosphate buffered saline.

### Example 3

### Timolol Microparticle In Vivo Study

A water-in-oil-in-water double emulsion (w/o/w) solvent evaporation technique was used for microsphere fabrication as previously described (Nihant, et al,, Journal of Colloid and Interface Science, 1995. 173(1): p. 55-65). Briefly, 40 mg of timolol maleate was dissolved in 300 μL of deionized water. Two hundred milligrams of the specific polymer was dissolved in 1 mL of dicholoromethane and 4 mL of trifluoroethanol. The aqueous and organic phases were emulsified via vortexing to obtain a desired active agent to polymer ratio of 10% or 20% (20 mg active agent/200 mg polymer or 40 mg active agent/200 mg polymer respectively). The emulsion was then added drop-wise to 200 mL of 5% (w/v) PVA aqueous solution and allowed to stir/harden for 3 hours. Microparticles were then collected by centrifugation, washed three times with deionized water, and freeze dried for 3 days.

The protocol follows the protocol outlined in Bertram, et al., (Journal of Microencapsulation, 2009. 26(1): p. 18-26) with one notable exception. The ratio of poly (D,L-lactic acid), Mn˜20-30 kDa to PLGA 502H is 30% PLA by weight to 70% PLGA by weight. The particles were administered to New Zealand White Rabbits.

Microspheres were brought to room temperature and resuspended in sterile PBS at a concentration of 25 mg/ml. Microspheres were delivered subconjunctivally by a single injection into the superior quadrant of both eyes of rabbits. Each injection contained 250 μl of the timolol microspheres solution. Injections were carried out using a 25 gauge needle and a 1 cc syringe.

IOP measurements in rabbit were taken using a Tonopen XL tonometer (Reichert Technologies, Depew, N.Y.). A series of at least three measurements was taken in awake animals after application of a topical anesthetic (proparicane, 0.5% solution). All IOP measurements were obtained between 10:00 and 11:00 PM.

Aqueous humor was collected weekly during the entire duration of the trial. 200 μl of aqueous humor was evacuated from the anterior chamber of one quarter of all eyes each week using a 28 gauge syringe needle. Serum samples were obtained from blood collected from the ear vein.

HPLC mass spectroscopy was used to analyze the serum samples, and no timolol was found at any time point.

Results indicate that IOP was significantly reduced compared to controls over the entire 80 day time course of the experiment.

Timolol was also detected in the aqueous humor using HPLC.

Additional in vivo release studies were carried out using triamcinolone after injection of microparticles at a concentration of 1 mg/ml (5 μl total) into rat eye, and methotrexate using the same concentration and injection protocol, as the triamcinolone (1 mg particles/ml PBS (5 μl total)). Triamcinolone was detected at a significant concentration 35 days after administration.

Numerous modifications and variations in the invention as set forth in the above illustrative examples are expected to occur to those skilled in the art. Consequently only such limitations as appear in the appended claims should be placed on the invention.

What is claimed is:

1. A composition comprising biodegradable microparticles comprising poly(D,L-lactic acid) (PLA) at a weight percent of less than 50% and a second polymer, the microparticles further comprising an ocular therapeutic agent, the microparticles having an average diameter of between about 5 microns and about 50 microns, the microparticles having a structure such that the therapeutic agent is released in a detectable amount from the microparticles for more than 90 days in vivo.

2. The composition of claim 1 wherein the microparticles having a structure such that the therapeutic agent is released in a detectable amount from the microparticles for more than 100 days in vivo, more than 120 days in vivo or more than 150 days in vivo.

3. The composition of claim 1 or 2 wherein the microparticles comprise PLA at a weight percent of at least 5%, at least 10%, at least 15%, at least 20%, at least 25%, at least 30%, at least 35%, at least 40%, or at least 45%.

4. The composition of claim 1, 2 or 3 wherein the PLA has a molecular weight of about 10 kDa to about 50 kDa, about 15 kDa to about 45 kDa, about 20 kDa to about 40 kDa, about 20 kDa to about 35 kDa, or about 20 kDa to about 30 kDa

5. The composition of claim 4 wherein the PLA has a molecular weight of about 25 kDa.

6. The composition of claim 1, 2, 3, 4 or 5 wherein the microparticles further comprise poly(lactic-co-glycolic acid) (PLGA).

7. The composition of claims 6 wherein the PLGA has a lactic acid to glycolic acid ratio of about 50:50 to about 75:25.

8. The composition of claim 6 or 7 wherein the PLGA is about 10 kDa, about 15 kDa, about 20 kDa, about 25 kDa, about 30 kDa, about 35 kDa, about 40 kDa, about 45 kDa, or about 50 kDa.

9. The composition of any one of claims 1-8 wherein the therapeutic agent is hydrophobic.

10. The composition of claim of any one of claims 1-8 wherein the ocular therapeutic agent is an agent that lowers intraocular pressure, an antibiotic, an inhibitor of a growth factor receptor, a chemotherapeutic agent, an anti-inflammatory agent or a steroid.

11. The composition of any one of claims 1-8 wherein the therapeutic composition is timolol.

12. The composition of any one of claims 1-8 wherein the therapeutic composition is methotrexate.

13. The composition of any one of claims 1-8 wherein the therapeutic composition is triamcinolone.

14. The composition of any of claims 1-13 which is injectable through a 30 gauge needle under clinically acceptable conditions of time and force.

15. The composition of any one of claims 1-14 wherein the microspheres comprise about 30% PLA and about 70% PLGA, the PLA has a molecular weight of about 25 kDa and the PLGA has a molecular weight of about 10 kDa, the PLGA has a lactic acid to glycolic acid ratio of about 50:50, and the therapeutic agent is hydrophobic.

16. A method of treating an ocular condition comprising the step of administering the composition of any one of claims 1-14 to a patient in an amount effective to treat the condition.

17. The method of claim 16 wherein the ocular condition is selected from the group consisting of glaucoma, diabetic retinopathy, age-related macular degeneration, and uveitis.

18. The method of claim 16 or 17 wherein administration is intravitreal or cubconjunctival.
